(19) United States
(12) Patent Application Publication
(10) Pub. No.: US 2016/0135776 A1
(43) Pub. Date: May 19, 2016

(54) METHOD AND SYSTEM FOR INTRAOPERATIVE IMAGING OF SOFT TISSUE IN THE DORSAL CAVITY

(71) Applicant: Howard C. CHANDLER, JR., Atlanta, GA (US)

(72) Inventor: Howard C. Chandler, JR., Atlanta, GA (US)

(21) Appl. No.: 14/897,218

(22) PCT Filed: Jun. 10, 2014
(86) PCT No.: PCT/US14/41768

§ 371 Date: Dec. 9, 2015

Related U.S. Application Data
(60) Provisional application No. 61/833,101, filed on Jun. 10, 2013.

(51) Int. Cl. A61B 6/00; G01R 33/48; G01R 33/56; A61B 6/03

(52) U.S. Cl. CPC A61B 6/5247; A61B 6/032; A61B 6/501; A61B 6/5217; G01R 33/4812; G01R 33/5608; A61B 2034/107

(57) ABSTRACT

The present invention provides methods and systems for the real-time imaging of the brain and other soft tissues within the dorsal cavity. The disclosed methods and systems have application in surgical procedures such as removal or resection of a pituitary tumor, a craniopharyngioma, a meningioma, an acoustic neuroma, an arachnoid cyst, an intraventricular tumor, a tumor located in the suprasellar cistern, a tumor located in a CSF filled intracranial cistern, a brain tumor, a cystic tumor, or a spinal tumor.

### CROSS-REFERENCE TO RELATED APPLICATIONS

The present application claims priority to U.S. Provisional Application No. 61/833,101, filed Jun. 10, 2013, the entire contents and disclosure of which are herein incorporated by reference thereto.

### TECHNICAL FIELD

This application relates to the field of intraoperative medical imaging. In particular, the present invention is directed to methods and systems that produce real-time images of the brain and other soft tissue within the dorsal cavity of body.

### BACKGROUND

In the field of medicine, it is often necessary to have an exact image of a body or tissue in order to plan and/or perform surgery or an irradiation treatment. Often, it is necessary to obtain the image as immediately before the irradiation and/or treatment as possible because a body or tissue can significantly change even within short periods of time. Currently, the ideal method for intraoperative imaging of tissue within the central nervous system (CNS) is an intraoperative MRI. An MRI provides greater delineation of different tissue elements in the CNS than do other methods such as a CT scan. A CT scan generally does not show images very well in the coronal and sagittal planes, which are the best for looking at brain tumors such as pituitary tumors, while an MRI does. However, an intraoperative MRI device is very expensive, and for many hospitals this option is cost prohibitive.

To obtain similar images as those produced by intraoperative MRI, various methods are under development to incorporate different imaging datasets. Separate images from CT scans, MRIs, and PET scans can be reconciled using a technique called image fusion. For example, with image fusion, a prior MRI image can be fused with a later CT scan, creating an MRI quality image based on the CT scan. This method is generally effective as long as there are no significant changes to the CNS tissues being imaged. However, the brain and other tissues often do change during surgery due to removal or resectioning of benign or malignant lesions and the movement of cerebrospinal fluid.

New software technology, called elastic image fusion, allows for image fusion even after certain changes have occurred in the brain. Elastic image fusion can be used when changes to the brain involve tumors that are inside the brain tissue and have similar elasticity to brain tissue. However, elastic image fusion is not effective when the changes to the brain involve tumors inside the cranial compartment that arise outside of the brain tissue. For example, elastic image fusion is not effective when operating on pituitary tumors. Pituitary tumors generally protrude upwards into the cisterns at the base of the brain. In the absence of a pituitary tumor, these cisterns are filled with cerebrospinal fluid (CSF). The object of the present invention is to overcome the above mentioned drawbacks by providing systems and methods for improved and more reliable intraoperative imaging of CNS tissue by allowing visualization of CSF and taking into account dynamic changes to tissue and CSF volumes within the CNS.

### SUMMARY

Aspects of the present invention relate to providing a superior and cost-effective alternative to the current cumbersome and expensive methods of obtaining real-time images of brain and tumor tissue involving repeated MRI scans in the operating room (OR) as the surgeon progressively removes the tumor and the brain shifts. Aspects of the present invention also overcome the shortcomings of CT images taken in the OR, which do not offer the exquisite delineation of different tissue elements in the brain that MRI provides and brain tumor surgery requires.

These aspects may comprise, and implementations may include, one or more or all of the components and steps set forth in the appended CLAIMS.

In a first aspect, the present invention is directed to a method for producing an enhanced image data set of an examination object within the dorsal cavity of a subject, the method comprising:
acquiring a magnetic resonance image (MRI) data set, determined by a magnetic resonance recording device, of the examination object at a first point in time, wherein the contours and three-dimensional volume of the examination object are delineated;
acquiring a computed axial tomography (CT) image data set, determined by a CT recording device, of the examination object at a second point in time, wherein the CT image data set is enhanced by a contrast agent;
acquiring a contour image data set which represents contours on the body of the subject in the form of points on the surface of the body substantially at the second point in time;
adapting the MRI data set to the CT image data set by taking into account the contour image data set to produce the enhanced image data set, wherein the enhanced image data set reveals changes in structure of soft tissue in the dorsal cavity; and
at least one of
   visualizing the enhanced image data set, and
   storing the enhanced image data set for later visualization.

In a second aspect, the present invention relates to an image processing system for producing an enhanced image data set of an examination object within the dorsal cavity of a subject, comprising:
an interface for receiving an MRI data set, determined by a magnetic resonance recording device, of the examination object at a first point in time, wherein the contours and three-dimensional volume of the examination object are delineated;
an interface to acquire a computed tomography (CT) image data set, determined by a CT recording device, of the examination object at a second point in time, wherein the CT image data set is enhanced by a contrast agent;
an interface to acquire a contour image data set which represents contours on the body of the subject in the form of points on the surface of the body substantially at the second point in time; and
an image fusion unit to adapt the MRI data set to the CT image data set by taking into account the contour image data set to produce the enhanced image data set, wherein the enhanced image data set reveals changes in structure of soft tissue in the dorsal cavity and
to at least one of
   visualize the enhanced image data set, and
   store the enhanced image data set for later visualization.

Particular implementations of the first aspect may include one or more or all of the following:
The MRI data set is adapted to the CT image data set by elastic image fusion.

The MRI data set is adapted to the CT image data set by subtracting a first volume from the examination object in the MRI data set, wherein the first volume is substantially equal to a second volume identified as contrasted cerebrospinal fluid (CSF) in the CT image set within the contours of the examination object.

The examination object is a tumor or a cyst.

The examination object is within a cranial compartment or a spinal compartment of the subject.

The enhanced image set is visualized in an MRI format which shows the degree of tumor resection and decompression of neurological structures in the axial, sagittal, and coronal planes.

The MRI data set is acquired before a surgical operation on the subject, and the CT image data set and contour image data set are acquired during the surgical operation.

The surgical operation may be a removal or resection of a pituitary tumor, a craniopharyngioma, a meningioma, an acoustic neuroma, an arachnoid cyst, an intraventricular tumor, a tumor located in the suprasellar cistern, a tumor located in a CSF filled intracranial cistern, a brain tumor, a cystic tumor, or a spinal tumor. The surgical operation may also comprise a partial resection of the tumor to prepare the tumor for postoperative radiosurgery and/or radiotherapy.

The contour image data set is obtained by laser-scanning the body, or by detecting markings or anatomical landmarks on the body, or from x-ray images containing markings attached onto the body.

The contrast agent is introduced into the CSF by intrathecal injection with a lumbar puncture or a ventricular catheter

Particular implementations of the second aspect may include one or more or all of the following:
a CT recording device along with the image processing system described in the second aspect.

A non-transitory computer program product configured to be loaded directly into a memory of the image processing system, including program code segments for executing all the steps of the method described in the first aspect of the invention when the program product is executed on the image processing system.

A non-transitory computer readable medium including program code segments when executed on a computer device of the image processing system, the program code segments causing the computer device to implement the method described in the first aspect of the invention.

Further, particular implementations of the second aspect may also include the particular implementations of the first aspect described above.

### BRIEF DESCRIPTION OF THE DRAWINGS

FIGS. 1A and 1B depict a pituitary tumor projecting upwards into the cisterns. Image is taken according to the systems and methods of the present invention.

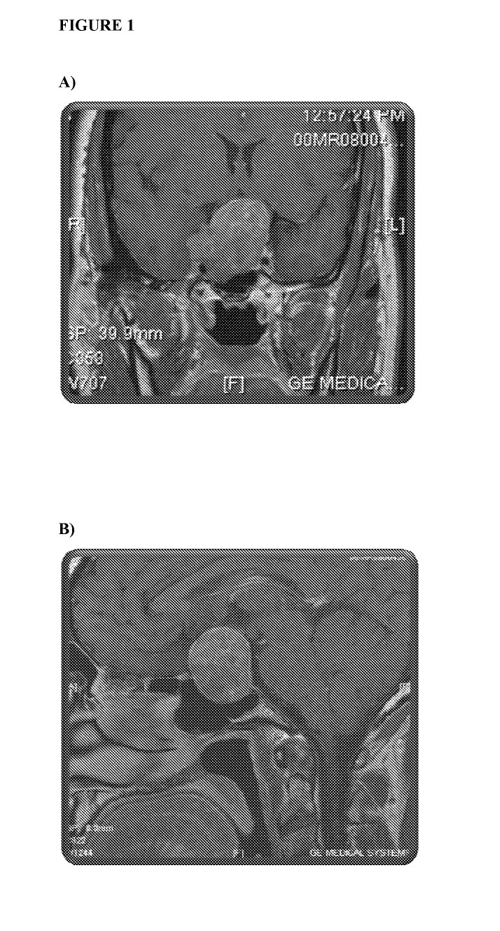

FIGS. 2A and 2B depict the same pituitary tumor progressively removed in the operating room. The top image was captured first, and the bottom was second. Image is taken according to the systems and methods of the present invention.

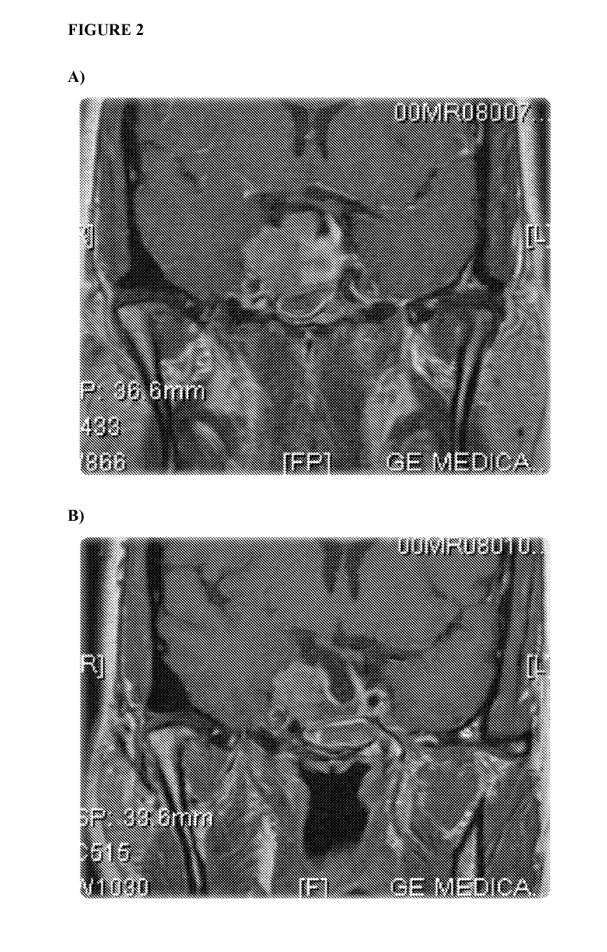

FIG. 3 depicts a flow diagram outlining an embodiment of the method for intraoperative imaging of soft tissue in the dorsal cavity such as a brain tumor.

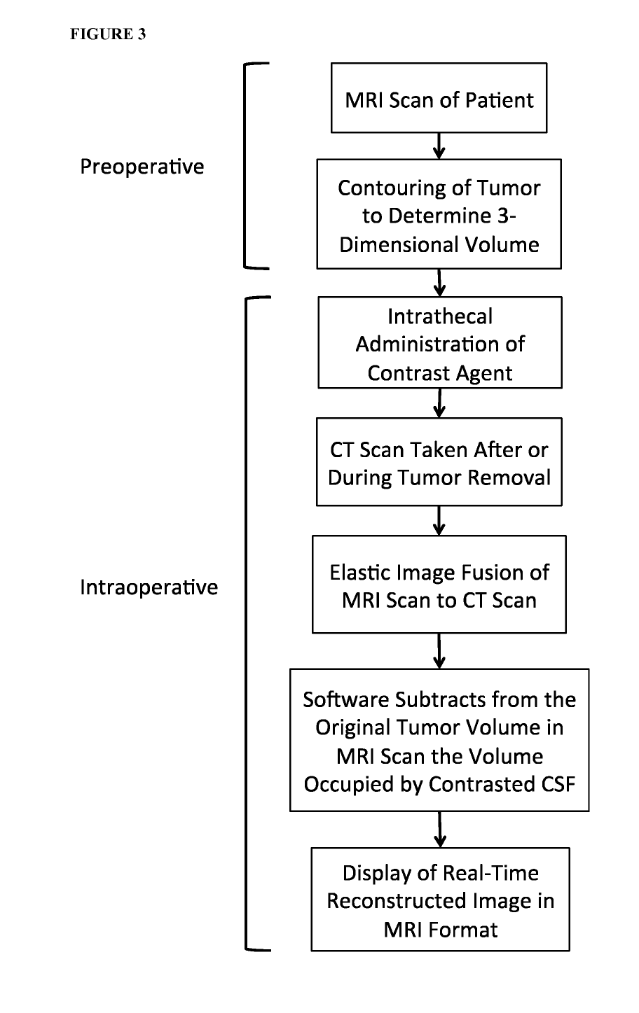

FIG. 4 depicts a flow diagram outlining a method for producing an enhanced image data set of an examination object within a dorsal cavity of a subject.

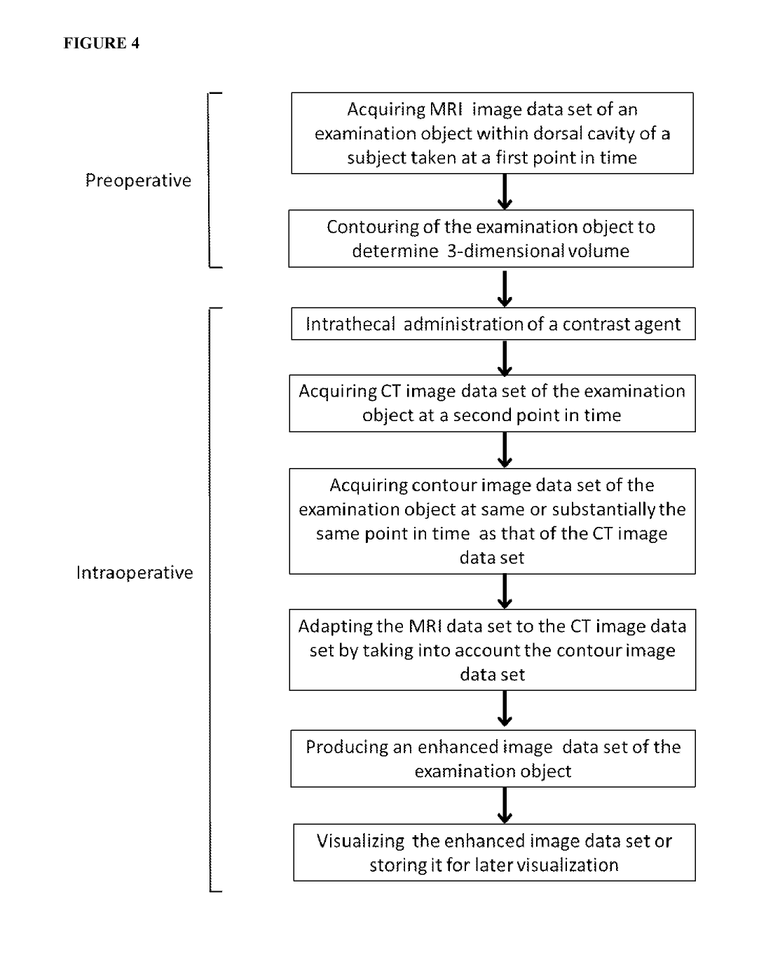

### DETAILED DESCRIPTION

The terminology used herein is for the purpose of describing particular embodiments only and is not intended to be limiting of the present invention. As used herein, the singular forms “a”, “an”, and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

As used herein, the verb “comprise” as is used in this description and in the claims and its conjugations are used in its non-limiting sense to mean that items following the word are included, but items not specifically mentioned are not excluded.

In the description below, particular implementations of the various embodiments of the present invention may include one or more or all of the various embodiments.

In one embodiment, the present invention is directed to a method for producing an enhanced image data set of an examination object within the dorsal cavity of a subject, the method comprising: acquiring a magnetic resonance image (MRI) data set, determined by a magnetic resonance recording device, of the examination object at a first point in time, wherein the contours and three-dimensional volume of the examination object are delineated; acquiring a computed axial tomography (CT) image data set, determined by a CT recording device, of the examination object at a second point in time, wherein the CT image data set is enhanced by a contrast agent; acquiring a contour image data set which represents contours on the body of the subject in the form of points on the surface of the body substantially at the second point in time; adapting the MRI data set to the CT image data set by taking into account the contour image data set to produce the enhanced image data set, wherein the enhanced image data set reveals changes in structure of soft tissue in the dorsal cavity; and at least one of visualizing the enhanced image data set, and storing the enhanced image data set for later visualization.

In one embodiment, the contours and three-dimensional volume of the examination object are be delineated by a surgeon or medical professional using navigation software. In another embodiment, the delineation of the contours and three-dimensional volume of the examination object is automated and produced by components of the image processing system disclosed herein.

In some embodiments, the examination object is within the dorsal cavity, the spinal compartment (i.e., the spinal cavity or vertebral canal), or the cranial compartment (i.e., the cranial cavity) of the subject.

In certain aspects, based on the MRI data set and the delineated contours a three-dimensional volume is calculated for the examination object. In other aspects, the CT image data set is analyzed to calculate the three-dimensional volume of cerebralspinal fluid occupying the location of the examination object in the MRI data set.

In other embodiments, the three-dimensional volumes of the CT and MRI image data sets may be defined by a stack of contours, each contour being defined on a corresponding plane parallel to a slice of the image volume. A contour is usually represented as a set of points, which may be interpolated to obtain closed contours. The voxels in the image volume may be masked by a 3D binary mask (i.e., a mask for each voxel in the 3D image volume). The 3D binary mask may be defined as a single-bit binary mask set having a single-bit mask for each voxel in the CT image volume or as a multi-bit mask set having a multi-bit mask for each voxel in the CT image volume. A single-bit binary mask can select or deselect voxels in the image volume to define a single three-dimensional volume. For example, the single bit value may be set to 1 for voxels that lie inside the three-dimensional volume defined by the contours and 0 for voxels that lie outside of the three-dimensional volume defined by the contours. A multi-bit mask allows multiple volumes of interest to be encoded in one 3D binary mask, with each bit corresponding to one three-dimensional volume.

The process described above may be automated by a segmentation tool or navigation software. The segmentation tool or navigation software may be used to manipulate a patient's medical image.

The segmentation tool may allow a user to delineate a volume of interest simultaneously from three cutting planes of the medical image: the axial plane, the sagittal plane, and the coronal plane. On the axial plane, a two-dimensional contour is displayed. The contour can be a solid contour when it is defined by a user or it can be a dashed-line contour interpolated from adjacent contours by a computer. A user can modify the contour by resizing it, scaling it or moving it. A user can also modify the shape of the contour by tweaking a shape morphing parameter. The shape morphing parameter defines how close the contour is to an ellipse. When the shape morphing parameter is set to 0, for example, the contour may be a standard ellipse. When the shape morphing parameter is set to 1, the contour may assume the outline of a spinal bone, for example, using automatic edge recognition methods as described, for example, in U.S. Pat. No. 7,327,865. By adjusting the morphing parameter in the range of [0, 1], the shape of the contour may be smoothly morphed from an ellipse, to a spinal bone, for example. A user can also adjust the shape of the contour, for example, using control points on a bounding box of the contour.

On the sagittal plane and coronal plane, a projected silhouette contour of the volume of interest may be displayed. The centers of all user-defined contours may be connected at the central axis of the spine, for example. A user can move, add or remove contours by moving or dragging the centers of the contours. When the center of a contour is moved on the sagittal or coronal planes, the actual contour defined on the axial image slice is moved accordingly. When the user selects any point in between two center points of adjacent axial contours, a new contour is added at that position, with the contour automatically set to the interpolation of the two adjacent axial contours. When a user drags and drops the center point of a contour outside the region of the two adjacent contours, or outside the image boundary, the contour is removed from the volume of interest. Once the volume of interest is delineated and stored in the geometrical format, it is converted to the volume format as a three-dimensional image volume containing only the voxels within the volume of interest. Other methods of defining the three-dimensional volume of an examination object similar to the one outlined above are also contemplated by the present invention. These methods may be applied to the original volume of the examination object shown in the MRI data set as well as to the volume occupied by the contrasted CSF in the CT image data set.

In some embodiments, the contrast agent used to enhance the CT image data set specifically delineates the CSF and its space and volume but not the other intracranial components.

Where data, regions, ranges or images are “acquired” this means that they are ready for use by the method in accordance with the invention. The data, regions, ranges or images can achieve this state of being “acquired” by for example being detected or captured (for example by analysis apparatuses) or by being input (for example via interfaces). The data can also have this state by being stored in a memory (for example a ROM, CD and/or hard drive) and thus ready for use within the framework of the method in accordance with the invention.

The data, regions, ranges or images can also be determined, in particular, calculated, in a method step before being acquired and/or before being stored.

In accordance with the method in accordance with the invention for producing an enhanced image data set, an MRI data set is provided which represents an image of a first region of a body, including at least a part of the surface of the body, at a first point in time. The MRI data set is preferably complete, but may no longer be up-to-date at the time of the treatment or surgical procedure. A second, CT image data set is also provided which represents an image of a second region of the body at a second point in time, wherein the first region and the second region overlap. The second point in time is later than the first point in time. A contour image data set is also provided which represents the contour of the body in the form of points on the surface of the body, substantially at the second point in time.

The wording “substantially at the second point in time” means that the point in time at which the contour image data set is obtained does not deviate at all or only slightly deviates from the second point in time. The difference in time between generating the contour image data set and the second point in time is significantly smaller, for example at most a twentieth, preferably at most a hundredth, of the difference in time between the first point in time and the second point in time. The CT image data set and the contour image data set are preferably prepared simultaneously or within a few minutes, while the period of time between the first point in time and the second point in time can be a number of hours, days or even weeks.

In one embodiment of the invention, the MRI data set is adapted to the CT image data set by elastic image fusion. The principle of elastic image fusion is known to the person skilled in the art of medical imaging. It is based on an iterative process in which the MRI data set is modified in steps and the modified image data set is compared with the CT image data set. Possible modifications include shifting, rotating or distorting the image data set and can be combined in any way. The surface of the body in the modified MRI data set is also referred to as a virtual contour. The comparison between the modified MRI data set and the CT image data set results in a degree of similarity which represents the similarity between the two image data sets. The modification of the MRI data set which results in the greatest degree of similarity results in a MRI data set which corresponds as well as possible to the CT image data set and thus best represents the current state of the body. This modification of the MRI data set is referred to herein as an “enhanced image data set”.

Many different algorithms are known from the prior art by which elastic image fusion can be implemented and optimized. One option is, for example, interpolation using thin-plate splines. A thin-plate spline interpolates a surface which is to remain unchanged at predetermined fixed points. This surface represents a thin metal plate which is deformed into the most economic shape in relation to the energy of deformation, i.e., the energy of deformation is minimized. Interpolation by means of thin-plate splines is, as with its derivatives, continuous in its own right, does not have any free parameters which have to be manually set, and features a closed solution.

In elastic image fusion, the contour image data set is, for example, taken into account by incorporating the distance between the points of the contour image data set and corresponding points in the MRI data set into the degree of similarity during image fusion. This means that the degree of similarity results not only from the image comparison between the modified MRI data set and the CT image data set but also from the distance between the surface of the body, which is represented by the contour image data set, and the surface in the modified MRI data set. This prevents the modified MRI data set from containing a virtual contour of the body which significantly deviates from the actual contour of the body, wherein it is possible for the distance by which the virtual contour in the modified MRI data set exceeds or falls short of the contour of the body represented by the contour image data set to be incorporated to varying degrees into the degree of similarity. Thus, a distance by which the virtual contour exceeds the actual contour of the body can for example reduce the degree of similarity more significantly than a comparable distance by which the virtual contour falls short of the actual contour.

Alternatively, no modifications of the MRI data set in which the first region represented by the modified MRI data set exceeds the contour of the body represented by the contour image data set are permitted during image fusion. This means that the contour image data set represents a firm boundary for possible modifications to the MRI data set. In an alternative embodiment, the MRI data set is adapted to the CT image data set in steps. In a first step, the MRI data set is adapted to the CT image data set without taking into account the contour image data set, wherein any adapting method can be used, including for example elastic image fusion. In a second step, the MRI data set which was adapted in the first step is segmented, wherein a first segment contains the overlap region between the first region and the second region, and a second segment contains the rest of the MRI data set.

This means that the first segment represents the region of the body which lies in the detection range of, for example, the CT device. All the data outside this region is contained in the second segment.

In a third step, this second segment of the MRI data set is adapted by taking into account the contour image data set. Various methods, for example, elastic image fusion, can also be used for adapting here. In this third step, the contour image data set is preferably taken into account by representing a boundary into which the second segment of the MRI data set is fitted, wherein the second segment is preferably adapted such that the transition to the first segment is continuous. This means that the second segment is not changed at the transition area to the first segment.

By adapting the MRI data set to the CT image data set in steps, the two segments of the MRI data set are separately optimized, thus achieving the best possible match between the MRI data set and the CT image data set in the overlap region between the first region and the second region, while the second segment is modified in accordance with the ancillary conditions provided by the contour image data set and thus represents the current contour of the body as well as possible.

In the case of elastic image fusion, a deformation field may be calculated which preferably contains a shift vector for the voxels (i.e., three-dimensional picture elements) of the MRI data set, in a three-dimensional matrix, wherein a shift vector can be provided for each voxel or only for some of the voxels. When taking into account the contour image data set, the deformation field is adapted to the contour in the contour image data set, for example, on the basis of corresponding contour control point pairs consisting of a surface point in the MRI data set and a corresponding surface point in the contour image data set. The deformation field is then applied to the MRI data set, wherein the data is for example interpolated by means of thin-plate splines. When the present invention is applied to two-dimensional image data, the matrix of the shift vectors is preferably also two-dimensional.

Two variants of adapting the MRI data set to the CT image data set are described in the following. In the first variant, a distance value is incorporated into the degree of similarity of elastic image fusion, in addition to the similarity between the modified MRI data set and the CT image data set. This distance value is determined from the distance between the points, which are represented by the contour image data set, and the surface of the body in the modified MRI image data set. Modifying the MRI image data set results in a new, virtual profile of the surface of the body in the modified MRI image data set. This modified MRI surface (or virtual contour) is compared with the contour of the body, as stored in the contour image data set on the basis of the points. The greater the distance between the modified surface in the modified MRI image data set and the surface in the contour image data set, the more significantly the degree of similarity resulting from the comparison between the modified MRI image data set and the CT image data set is reduced. This can reach the point where the degree of similarity is reduced to zero, if some or all of the points in the modified MRI image data set lie within the body, i.e. the virtual contour of the body in the modified MRI image data set extends beyond the measured surface of the body represented by the contour image data set, at the second point in time. In this case, the contour image data set serves as a firm boundary for possible modifications to the MRI image data set during elastic image fusion.

The distance value is for example the sum of the distances between the individual points and the virtual contour in the modified MRI image data set or its average value. The distance is for example the minimum distance between a point and the virtual contour or the distance from a corresponding point, for example a landmark. A landmark is a defined, characteristic point of an anatomical structure which is always identical or recurs with a high degree of similarity in the same anatomical structure of a number of patients. Typical landmarks are for example the epicondyles of a femoral bone, the tips of the transverse processes and/or dorsal process of a vertebra or points such as the tip of the nose or the end of the root of the nose.

In accordance with a second variant, the MRI image data set is adapted to the CT image data set in steps. In a first step, the MRI data set is adapted to the CT image data set, for example by means of elastic image fusion, without taking into account the contour image data set. The result of this is that the adapted MRI data set and the CT image data set are optimally matched in the overlap region after the first step. The MRI data set which was adapted in the first step is then segmented, such that the first segment represents the overlap region and a second segment contains the rest of the adapted MRI data set. In a third adapting step, the second segment of the MRI data set is adapted in the region to be supplemented. The second segment of the MRI data set is for example adapted such that it is optimally fitted into the region which is limited on the one hand by the boundary of the first segment of the modified MRI data set and on the other hand by the points in the contour image data set. In this step, it is possible to take into account the ancillary condition that the transition from the first segment to the second segment of the MRI data set should run continuously, i.e., the data of the second segment which immediately borders the first segment is not changed or only slightly changed.

The advantage of the second variant is that the MRI data set is optimally matched to the CT image data set in the detection range of the CT recording device, while the MRI data set is simultaneously optimally fitted into the contour of the body represented by the contour image data set. The contour image data set is for example obtained by laser-scanning the surface or a part of the surface of the body. One possible method for laser-scanning is described in detail in European Patent Application EP 1 142 536 A1, wherein the surface of the body which is to be captured is moved into the detection range of a navigation system which is assisted by at least two cameras and captures the three-dimensional spatial locations of light markings with computer assistance. Light markings are generated on the surface of the body to be referenced by means of a light beam, preferably a tightly focused laser beam, and their three-dimensional location is determined by the camera-assisted navigation system. The location of the light marking is stereoscopically calculated from the locations and alignments of the cameras and from the images generated by them. Optionally, additional information concerning the distance of the light marking from a camera is ascertained from the size of the light marking in the image of the camera. The three-dimensional locations of the scanned surface points are combined to form the contour image data set.

Alternatively, it is possible to project a grid of laser beams onto the surface of the body, capture the grid by means of at least two cameras, and calculate locations of surface points from this, which are stored in the contour image data set.

Alternatively or additionally, the contour image data set is obtained by detecting markings on the body. Such a marking can for example be a marker or a marker device.

It is the function of a marker to be detected by a marker detection device (for example, a camera or an ultrasound receiver), such that its spatial position (i.e., its spatial location and/or alignment) can be ascertained. Such markers can be active markers. An active marker emits for example electromagnetic radiation and/or waves, wherein said radiation can be in the infrared, visible and/or ultraviolet spectral range. The marker can also however be passive, i.e., it can for example reflect electromagnetic radiation from the infrared, visible and/or ultraviolet spectral range. To this end, the marker can be provided with a surface which has corresponding reflective properties. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can also, however, exhibit a cornered (for example, cubic) shape.

Furthermore, the image contour data set is alternatively or additionally obtained from x-ray images which contain markings attached on the body. The x-ray images generated by a computed tomograph when ascertaining the CT image data set can for example be used for this purpose. Thus, no additional hardware is necessary in order to obtain the image contour data set. The markings, preferably in the form of small metal plates or metal spheres, are attached on the body and visible in the individual x-ray images of the CT. Another option is to integrate markings, preferably metallic markings, into an item of clothing which lies tightly against the body.

Alternatively, the image contour data set can also be obtained by scanning the surface of the body by means of a pointer, wherein the tip of the pointer is placed onto various points of the surface of the body and the location of the tip is ascertained.

A pointer is a rod comprising one or more—advantageously, two—markers fastened to it, wherein the pointer can be used to measure off individual coordinates, in particular spatial coordinates (i.e. three-dimensional coordinates), on a body, wherein a user guides the pointer (in particular, a part of the pointer which has a defined and advantageously fixed position with respect to the at least one marker which is attached to the pointer) to the location corresponding to the coordinates, such that the location of the pointer can be determined by using a surgical navigation system to detect the marker on the pointer. The relative position between the markers of the pointer and the part of the pointer used to measure off coordinates (in particular, the tip of the pointer) is in particular known. The surgical navigation system then enables the location (the three-dimensional coordinates) of the part of the pointer contacting the body and therefore the contacted point on the surface of the body to be calculated, wherein the calculation can be made automatically and/or by user intervention.

The present invention also relates to a non-transitory computer program which, when it is run on a computational unit, performs one or more of the method steps described herein.

Within the framework of the invention, non-transitory computer program elements can be embodied by hardware and/or software (this also includes firmware, resident software, micro-code, etc.). Within the framework of the invention, non-transitory computer program elements can take the form of a non-transitory computer program product which can be embodied by a computer-usable or computer-readable storage medium comprising computer-usable or computer-readable program instructions, “code” or a “computer program” embodied in said medium for use on or in connection with the instruction executing system. Such a system can be a computer; a computer can be a data processing device comprising means for executing the computer program elements and/or the program in accordance with the invention. Within the context of this invention, a computer-usable or computer-readable medium can be any medium which can contain, store, communicate, propagate or transport the program for use on or in connection with the instruction executing system, apparatus or device. The computer-usable or computer-readable medium can for example be, but is not limited to, an electronic, magnetic, optical, electromagnetic, infrared or semiconductor system, apparatus, device or medium of propagation, such as for example the Internet. The computer-usable or computer-readable medium could even for example be paper or another suitable medium onto which the program is printed, since the program could be electronically captured, for example by optically scanning the paper or other suitable medium, and then compiled, interpreted or otherwise processed in a suitable manner.

In a further embodiment, the method of the present invention comprises one or more of the following steps:
1) Preoperative MRI scan acquisition in a brain tumor patient
2) Preoperative contouring of tumor by a surgeon using cranial navigation software
3) Intrathecal administration of a contrast agent by lumbar puncture or ventricular catheter
4) Intraoperative CT scan taken after or during tumor removal
5) Elastic image fusion of the preoperative MRI to the intraoperative CT
6) Software subtracts from delineated original tumor volume wherever contrasted spinal fluid is detected in the three dimensional space the tumor was occupying preoperatively
7) Real time reconstructed image is then displayed in MRI format showing degree of tumor resection and decompression of neurological structures in axial, sagittal and coronal planes.

In further embodiments, the methods and systems of the present invention can be used for pituitary tumors, craniopharyngioma, meningioma, acoustic neuroma, arachnoid cysts, intraventricular tumors, tumors located in the suprasellar cistern, tumors located in any CSF filled intracranial cisterns, endoscopic resections of brain tumors, image-guided aspiration of cystic tumors, and spinal tumors.

In still other embodiments, the present invention may be used in conjunction with “adaptive hybrid surgery.” “Adaptive hybrid surgery” occurs where during image guided tumor resections cranial navigation system allows the surgeon to perform an intended and pre-planned partial resection of the tumor in order to get the tumor to an ideal size and shape for a subsequent planned postoperative radiosurgery/radiotherapy treatment. In certain aspects, the present invention is critical to using an adaptive hybrid surgery technique in pituitary tumors with an intraoperative CT scanner. Thus, the disclosed methods may further comprise intraoperative radiosurgery planning and/or execution.

The present invention contemplates using intrathecal injection of a radiographic contrast agent prior to surgery with the use of intraoperative CT scanning during or after tumor removal to generate a real time dataset, then performing image fusion with an MRI from the patient, preferably with elastic image fusion, and using a software algorithm that subtracts from the preoperative MRI tumor volume anywhere CSF filled with contrast agent is detected. Advantageously, such a technique or method adequately assesses the regression or withdrawal of the tumor from the brain structures it is impinging upon. Importantly, the image displayed to the surgeon can be a representation of an MRI in both the coronal and sagittal planes. In certain embodiments, the present invention is used for menigiomas, acoustic neuromas, other skull base tumors, and spinal tumors.

In another embodiment, one or more of the image datasets is stored in a cloud database. This allows sharing of the image datasets while at the same time reducing the demand for local storage space.

According to another embodiment, the enhanced image dataset is adapted in a cloud. This reduces the demand for computational power on a local machine on which the method is performed. This accelerates the generation of the enhanced image datasets without computational constraints.

The method in accordance with the invention is in particular a data processing method. The data processing method is preferably performed using technical means, in particular a computer. In particular, the data processing method is executed by or on the computer. The computer in particular comprises a processor and a memory in order to process the data, in particular electronically and/or optically. The adapting steps described are in particular performed by a computer. A computer is in particular any kind of data processing device, in particular, an electronic data processing device. A computer can be a device which is generally thought of as such, for example desktop PCs, notebooks, netbooks, etc., but can also be any programmable apparatus, such as for example a mobile phone or an embedded processor. A computer can in particular comprise a system (network) of “sub-computers”, wherein each sub-computer represents a computer in its own right.

The term of computer encompasses a cloud computer, in particular a cloud server, The term of cloud computer encompasses cloud computer system in particular comprises a system of at least one cloud computer, in particular plural operatively interconnected cloud computers such as a server farm. Preferably, the cloud computer is connected to a wide area network such as the world wide web (WWW). Such a cloud computer is located in a so-called cloud of computers which are all connected to the world wide web. Such an infrastructure is used for cloud computing which describes computation, software, data access and storage services that do not require end-user knowledge of physical location and configuration of the computer that delivers a specific service. In particular, the term “cloud” is used as a metaphor for the interact (world wide web). In particular, the cloud provides computing infrastructure as a service (IaaS). The cloud computer may function as a virtual host for an operating system and/or data processing application which is used for executing the inventive method.

A computer in particular comprises interfaces in order to receive or output data and/or perform an analogue-to-digital conversion. The data are in particular data which represent physical properties and/or are generated from technical signals. The technical signals are in particular generated by means of (technical) detection devices such as for example devices for detecting marker devices) and/or (technical) analytical devices such as for example devices for performing imaging methods), wherein the technical signals are in particular electrical or optical signals. The technical signals represent in particular the data received or outputted by the computer.

In one embodiment of the invention, the image processing system also comprises a radiotherapy device which for example contains a LINAC (linear accelerator) and can be controlled on the basis of the enhanced image data set. A treatment plan can be derived from the enhanced image data set, on the basis of which the radiotherapy device is configured and activated.

The therapy beam generated by the radiotherapy device can optionally be used for imaging, i.e., in particular for generating the CT image data set. The therapy beam usually exhibits a higher energy level than an x-ray beam, for example in the megavolt (MV) range as compared to the kilovolt (kV) range in the case of x-ray radiation.

Preferably, at least the CT recording device for generating the CT image data set is arranged on a support, which is also referred to as a gantry, wherein the support can be rotationally and/or translationally moved, for example with respect to a table on which the body is situated. Other devices, such as the device for generating the contour image data set or the radiotherapy device, or components of the devices are optionally arranged on the same support. The position of the devices and/or components relative to each other are thus known, and the position of the devices and/or components with respect to the body can be changed by moving a single support.

Finally, it may be pointed out once again that the method previously described in detail and the system architecture are only preferred exemplary embodiments that can be modified by the person skilled in the art in the most varied ways without departing from the scope of the invention to the extent it is prescribed by the claims.

Further, elements and/or features of different example embodiments may be combined with each other and/or substituted for each other within the scope of this disclosure and appended claims.

Still further, any one of the above described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks, cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A method for producing an enhanced image data set of an examination object within a dorsal cavity of a subject, the method comprising:
acquiring a magnetic resonance image (MRI) data set, determined by a magnetic resonance recording device, of the examination object at a first point in time, wherein the contours and three-dimensional volume of the examination object are delineated;
acquiring a computed axial tomography (CT) image data set, determined by a CT recording device, of the examination object at a second point in time, wherein the CT image data set is enhanced by a contrast agent;
acquiring a contour image data set which represents contours on the body of the subject in the form of points on the surface of the body substantially at the second point in time;
adapting the MRI data set to the CT image data set by taking into account the contour image data set to produce the enhanced image data set, wherein the enhanced image data set reveals changes in structure of soft tissue in the dorsal cavity; and
at least one of
visualizing the enhanced image data set, and
storing the enhanced image data set for later visualization.

2. The method according to claim 1, wherein the MRI data set is adapted to the CT image data set by elastic image fusion.

3. The method according to claim 2, wherein the MRI data set is adapted to the CT image data set by subtracting a first volume from the examination object in the MRI data set, wherein the first volume is substantially equal to a second volume identified as contrasted cerebrospinal fluid (CSF) in the CT image set within the contours of the examination object.

4. The method of claim 3, wherein the examination object is a tumor or a cyst.

5. The method of claim 4, wherein the enhanced image set is visualized in an MRI format which shows the degree of tumor resection and decompression of neurological structures in the axial, sagittal, and coronal planes.

6. The method of claim 4, wherein the MRI data set is acquired before a surgical operation on the subject, and
the CT image data set and contour image data set are acquired during the surgical operation.

7. The method of claim 6, wherein the surgical operation is a removal or resection of a pituitary tumor, a craniopharyngioma, a meningioma, an acoustic neuroma, an arachnoid cyst, an intraventricular tumor, a tumor located in the suprasellar cistern, a tumor located in a CSF filled intracranial cistern, a brain tumor, a cystic tumor, or a spinal tumor.

8. The method of claim 6, wherein surgical operation comprises a partial resection of the tumor to prepare the tumor for postoperative radiosurgery and/or radiotherapy.

9. The method according to claim 1, wherein the examination object is within a cranial compartment or a spinal compartment of the subject.

10. The method according to claim 1, wherein the contour image data set is obtained by laser-scanning the body.

11. The method according to claim 1, wherein the contour image data set is acquired by detecting markings or anatomical landmarks on the body.

12. The method according to claim 1, wherein the contour image data set is acquired from x-ray images containing markings attached onto the body.

13. The method according to claim 1, wherein the contrast agent is introduced into the CSF by intrathecal injection with a lumbar puncture or a ventricular catheter.

14. An image processing system for producing an enhanced image data set of an examination object within a dorsal cavity of a subject, comprising:
an interface for receiving an MRI data set, determined by a magnetic resonance recording device, of the examination object at a first point in time, wherein the contours and three-dimensional volume of the examination object are delineated;
an interface to acquire a computed tomography (CT) image data set, determined by a CT recording device, of the examination object at a second point in time, wherein the CT image data set is enhanced by a contrast agent;
an interface to acquire a contour image data set which represents contours on the body of the subject in the form of points on the surface of the body substantially at the second point in time; and
an image fusion unit to adapt the MRI data set to the CT image data set by taking into account the contour image data set to produce the enhanced image data set, wherein the enhanced image data set reveals changes in structure of soft tissue in the dorsal cavity and
to at least one of
visualize the enhanced image data set, and
store the enhanced image data set for later visualization.

15. The image processing system for producing an enhanced image data set of an examination object within the dorsal cavity of a subject according to claim 14, further comprising:
a CT recording device; and
an image processing system according to claim 11.

16-31. (canceled)

32. A non-transitory computer readable medium including program code segments when executed on a computer device of an image processing system for producing an enhanced image data set of an examination object within a dorsal cavity of a subject, the program code segments causing the computer device to implement a method comprising:
acquiring a magnetic resonance image (MRI) data set, determined by a magnetic resonance recording device, of the examination object at a first point in time, wherein the contours and three-dimensional volume of the examination object are delineated;
acquiring a computed axial tomography (CT) image data set, determined by a CT recording device, of the examination object at a second point in time, wherein the CT image data set is enhanced by a contrast agent;
acquiring a contour image data set which represents contours on the body of the subject in the form of points on the surface of the body substantially at the second point in time;
adapting the MRI data set to the CT image data set by taking into account the contour image data set to produce the enhanced image data set, wherein the enhanced image data set reveals changes in structure of soft tissue in the dorsal cavity; and
at least one of
visualizing the enhanced image data set, and
storing the enhanced image data set for later visualization.

33. The non-transitory computer readable medium according to claim 32, wherein the MRI data set is adapted to the CT image data set by subtracting a first volume from the examination object in the MRI data set, wherein the first volume is substantially equal to a second volume identified as contrasted cerebrospinal fluid (CSF) in the CT image set within the contours of the examination object.

34. The non-transitory computer readable medium according to claim 32, wherein the enhanced image set is visualized in an MRI format which shows the degree of tumor resection and decompression of neurological structures in the axial, sagittal, and coronal planes.

35. The non-transitory computer readable medium according to claim 32, wherein the MRI data set is acquired before a surgical operation on the subject, and
the CT image data set and contour image data set are acquired during the surgical operation.

36. The non-transitory computer readable medium according to claim 35, wherein the surgical operation is a removal or resection of a pituitary tumor, a craniopharyngioma, a meningioma, an acoustic neuroma, an arachnoid cyst, an intraventricular tumor, a tumor located in the suprasellar cistern, a tumor located in a CSF filled intracranial cistern, a brain tumor, a cystic tumor, or a spinal tumor.
